(19) United States
(12) Patent Application Publication
(10) Pub. No.: US 2013/0172960 A1
(43) Pub. Date: Jul. 4, 2013

(54) METHOD FOR LOW-LEVEL LASER IRRADIATION FOR HEARING LOSS RESTORATION

(71) Applicants: Jin Chul Ahn, Gyeonggi-do (KR); Chung Ku Rhee, Chungcheongnam-do (KR); Myung Whan Suh, Seoul (KR)

(72) Inventors: Jin Chul Ahn, Gyeonggi-do (KR); Chung Ku Rhee, Chungcheongnam-do (KR); Myung Whan Suh, Seoul (KR)

(73) Assignee: INDUSTRY-ACADEMIC COOPERATION FOUNDATION, DANKOOK UNIVERSITY, Gyeonggi-do (KR)

(21) Appl. No.: 13/728,146

(22) Filed: Dec. 27, 2012

(30) Foreign Application Priority Data
Dec. 28, 2011 (KR) 10-2011-0145103

(51) Int. Cl. A61F 11/00

(52) U.S. Cl. CPC A61F 11/00
USPC 607/89

(57) ABSTRACT

A method for low-level laser irradiation for hearing loss reversal includes: generating low-frequency laser light; controlling an output intensity of the generated laser light; determining whether or not the wavelength and output of the laser light coincides with a preset reference wavelength and reference output intensity, respectively; and irradiating with the laser light into an inner ear through an external auditory meatus, wherein the reference wavelength is set in the range of 600 nm to 10,000 nm.

### BACKGROUND OF THE INVENTION

The present invention relates to a method for low-level laser irradiation for hearing loss restoration, and more particularly, to a method for low-level laser irradiation which is capable of reversing noise induced hearing loss (NIHL) in auditory hair cells of a human body exposed to a noise generating area, or particularly, in auditory hair cells within a cochlear tissue.

In general, the sense of hearing, being one of the abilities possessed by human beings used to perceive the environment, is a very important, sense used as a means for collecting information through hearing as a supplement to visible perception. Therefore, the sense of hearing is very important for human life. Hearing loss is divided into sudden sensorineural hearing loss (sudden hearing loss) and natural hearing loss. Sudden hearing loss refers to hearing loss in which hearing suddenly decreases, and natural hearing loss refers to hearing loss in which hearing gradually decreases for senescent reasons. In the case of sudden hearing loss in which hearing suddenly decreases, it is important to discover the cause of the sudden hearing loss and treat the sudden hearing loss in the early stages.

In modern medicine, lasers are used in various forms. Conventionally, the medical applications of lasers have been limited to surgery. Recently, however, the medical applications of lasers have been widely expanded to non-vascular diagnosis and therapies. Initially, the laser was introduced to surgical procedures. The laser introduced into the surgery field has a high output of energy and has been used for tissue destroying operations. In the tissue destroying operation, the high-level laser has been applied to tissue cutting, clotting, hemostasis and the like. Then, low-level laser therapy has been applied to wound care, and research has been conducted on the clinical performance and photobiological and photochemical reactions of low-level lasers. The research has reported that low-level lasers have a tissue repair effect, a pain relief effect, an anti-inflammatory action, an anti-edema effect, a systemic vascular change effect, a tissue stimulating effect and the like. Thus, low-level lasers are widely used in various fields of medicine. For example, low-level lasers are used for treating a wound, a damaged tissue and the like in dermatology, an inner ear disease, a middle ear infection and the like in otolaryngology, bruising, a fracture injury and the like in acupuncture, aphthous stomatitis, gingivitis and the like in dentistry, and rheumatoid arthritis and the like in orthopedics.

Meanwhile, a photochemical reaction for plants can be found in carbon assimilation. It is known that when light is irradiated on a living body, APT is produced by the photochemical reaction and activates tissues. That is, when a low-level laser having such a level as to not damage living cells is irradiated on a living body, a physiological function of the living body is activated, and spontaneous curing ability in an affected area is improved. This fact has been well known in the medical fields through various experiments during surgical operations using high-level lasers. Recently, a low-level laser therapy (LLLT) for treating various diseases having the above-described effects has been developed and applied to various diseases.

The biochemical reaction of light is determined according to the wavelength, polarization, and intensity of the light. An experiment has reported that light having an energy of about 100 mV as polarized light having a range of 600 um to 1,000 um has the greatest effect on the body. The light may be generated by a He—Ne laser, semiconductor laser, YAG laser or the like. In particular, the semiconductor laser has an advantage in terms of size or price. Currently, a semiconductor laser based on GaAs (904 nm), GaAlAs (780-820-870 nm), or InGaAlP (630-685 nm) has been developed and launched on the market.

The LLLT is now actively used for various pain treatments. The LLLT has been significantly developed after Dr. Plog in Canada achieved a great effect in controlling pain by irradiating a He—Ne laser in the form of laser acupuncture onto acupoints.

Furthermore, research has been conducted on the clinical performance and the photobiological and photochemical reactions of low-level lasers, and have reported that low-level lasers have a tissue rep fair effect, a pain relief effect, an anti-inflammatory action, an anti-edema effect, a systemic vascular change effect, a tissue stimulating effect and the like. Accordingly, low-level lasers are being used in various medical fields.

As the related art, Korean Patent Laid-open Publication No. 10-2009-0118122 discloses a medical device for treatment of an ear ailment that treats or relieves the symptoms of the disease by using optical properties based on human auditory characteristics. The applicable range of the light source may include a visible ray range and a near-infrared ray range. And the light source is a laser including a light emitting diode (LED) and a laser diode (LD). The ear ailment treatment may selectively irradiate with the wavelength as selected depending on the symptoms of a disease, or stably irradiate the white light which, is obtained by mixing three colours in a predetermined ratio by controlling the cycle and duration of the light according to the order of being stored in the memory of the device The ear ailment cure has not undergone clinical trials on how a laser light may be irradiated deeper into the body. Therefore, the effect or side effect of the ear ailment cure is unknown. Furthermore, information on the frequency, power, and irradiation time of the laser in obtaining an effect is unknown, and information on reactions by which a treatment may be stopped or delayed is unknown. Accordingly, there are difficulties in applying the ear ailment cure to actual treatments.

### SUMMARY OF THE INVENTION

An embodiment of the present invention is directed, to a method of low-level laser irradiation for hearing loss reversal, which irradiates a specific frequency of low-level light through an external auditory meatus and eardrum and induces activation of auditory hair cells, thereby confirming whether an inner ear disease has been cured or not.

In accordance with an embodiment of the present invention, a method, for low-level laser irradiation for hearing loss reversal includes: generating low-frequency laser light; controlling an output intensity of the generated laser light; determining whether or not the wavelength and output of the laser light coincide with a preset reference wavelength and reference output intensity, respectively; and irradiating the laser light into an inner ear through an external auditory meatus, wherein the reference wavelength is set in the range of 700 nm to 1000 nm.

The reference wavelength may include any one selected from an infrared light wavelength group consisting of 780 nm, 830 nm, and 980 nm.

The reference output intensity may be set in the range of 50 mW to 300 mW.

The reference output intensity may be set to 165 mW.

The laser light irradiation may include periodically irradiating the generated laser light for 10 to 60 minutes per day.

The laser light irradiation may include irradiating with the generated laser light for one to 14 days.

The method may further include checking a hearing threshold at a plurality of frequencies, after the laser light irradiation. Meanwhile, the hearing may be checked every day.

The checking of the hearing threshold may include checking the hearing threshold, at one or more irradiation frequencies selected from an irradiation frequency group consisting of 0.25 kHz, 0.5 kHz, 1 kHz, 2 kHz, 4 kHz, and 8 kHz.

### BRIEF DESCRIPTION OF THE DRAWINGS

FIG. 1 is a flow chart showing a process of laser-irradiation according to an embodiment of the present invention.

FIGS. 2 to 4 are graphs showing changes in the hearing threshold of an experimental object onto which representative lasers in an infrared wavelength band are irradiated.

FIG. 5 is a graph comparatively showing hearing threshold data of a treated group and an untreated group at each frequency before noise exposure.

FIG. 6 is a graph comparatively showing hearing threshold data after noise exposure at each frequency after laser irradiation according to the embodiment of the present invention is performed.

### DESCRIPTION OF SPECIFIC EMBODIMENTS

Exemplary embodiments of the present invention will be described in detail with reference to the accompanying drawings so that they can be readily implemented by those skilled in the art. The drawings are not necessarily to scale and in some instances, proportions may have been exaggerated in order to clearly illustrate features of the embodiments. Furthermore, terms to be described below have been defined by considering functions in embodiments of the present invention, and may be defined differently depending on a user or operator's intention or practice. Therefore, the definitions of such terms are based on the overall descriptions of the present specification.

The embodiments of the present invention provide a method capable of efficiently improving hearing loss reversal through a process of lowering a hearing threshold of a damaged auditory hair cell while a specific frequency of laser is irradiated onto the auditory hair cell for a predetermined period.

### Embodiments

Low-Level Laser Irradiation Method

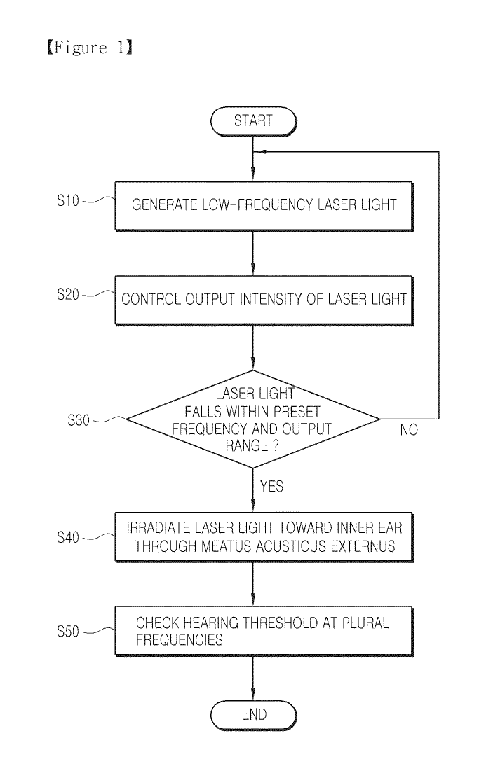

Hereinafter, referring to FIG. 1, a process of laser-irradiation according to an embodiment of the present invention will be described.

First, a low-frequency laser light is generated from a light source at step S10. In this embodiment of the present invention, infrared light having a wavelength range of 600 to 1000 nm may be used as the laser light. Specifically, the laser light may be required to maintain the wavelength range of 600 to 1000 nm, in order to increase the therapeutic efficiency.

After step S10, the generated laser light may be controlled to a required output, level through a separate output controller at step S20. The required output level may range from 50 to 300 mW/cm². Desirably, the required output level may be maintained at 165 mW/cm².

After step S20, whether or not the generated laser light falls within the preset wavelength range and coincides with the required output level is determined at step S30. When it is determined at step S30 that the generated laser light does not fall within the preset wavelength range and does not coincide with the required output level, the process returns to step S10. Then, the wavelength of laser light generated from the light source is controlled at step S30.

When it is determined at step S30 that the generated laser light falls within the preset wavelength, range and coincides with the required output level, the low-frequency laser light is irradiated toward the inner ear through the meatus acusticus externus and eardrum at step S40. During this process, a laser light transmission module such as optical fiber may be used. At step S40, a plurality of frequency steps and repetitive irradiations are performed.

After step S40, hearing thresholds are checked in a plurality of frequency domains at step S50.

Hereinafter, results obtained by testing experimental objects through a proper testing device will be described, in order to specifically prove the hearing loss restoration effect according to the embodiment of the present invention.

Object for Measurement

As experimental objects for the present, invention, cultured rats (SD rats) having a weight of about 200 g are selected. The SD rats are divided into a noise only group and a noise and laser group. The SD rats are anesthetized before the recording of auditory brainstem response (ABR) and light irradiation. The SD rats are sacrificed after a 12-day long treatment throughout the scanning electron microscope (SEM) research.

Noise Exposure and Laser Irradiation

A noise box made of an acrylic material and having a speaker attached at the top thereof is provided to expose the SD rats to noise. A dual-channel real-time frequency analyzer is used, to correct a noise generator. Furthermore, it is checked whether or not an amplifier projects settings from the noise generator accurately. During the experiment, experiment interference may occur. For example, the SD rats' ears may be covered inside the noise box. Therefore, each of the SD rats is placed in a small and separated space such that the experiment interference is prevented. The SD rats serving as experimental objects are once exposed for 6 hours to a narrow-band, noise of 120 dB SPL which, is concentrated at a frequency of 16 kHz.

For the experimental objects belonging to the noise and laser exposure group, laser light is irradiated at an energy output of 165 mW/cm² by 780 nm, 830 nm, and 980 nm diode lasers for 60 minutes per day during 12 days. In particular, it is desirable that the laser light irradiation is done with a 830 nm diode laser. Here, the diode laser irradiation time may be varied within the range of 10 to 60 minutes, and the number of days during which the laser light irradiation takes place may be varied within the range of 1 to 14 days.

The energy output from the diode laser is 165 mW, and an output transmitted to the inner ear is measured at 10.24±2.04 mW, corresponding to 6.20±1.24% of the initial output.

The hearing thresholds of the experimental objects are measured at 4 kHz, 8 kHz, 12 kHz, 16 kHz, and 32 kHz before the noise exposure. After the noise exposure, measurements for recording the restoration of hearing threshold are performed at first, fifth, tenth, and twelfth irradiations, respectively.

Meanwhile, hearing thresholds for the human body may be checked at one or more irradiation frequencies selected from an irradiation frequency group consisting of 0.125 kHz, 0.25 kHz, 0.5 kHz, 1 kHz, 2 kHz, 3 kHz, 4 kHz, 8 kHz, 12 kHz, and 16 kHz.

Auditory Brainstem Response Recording

Auditory brainstem response (ABR) recording is performed by a signal processing system including a tone burst generator and frequency specific stimulus generation modules.

The experimental objects are placed in a soundproof booth, and three electrodes are inserted into the subcutaneous tissue of the ear of each experimental object. One electrode is disposed at the top, and the two other electrodes are disposed on the side surfaces. More specifically, the three electrodes may be disposed under the auricle, and may be referred to as an active electrode, a reference electrode, and a ground electrode, respectively.

A tone burst auditory stimulus is transmitted to a tube inserted into the external ear of the experimental object, and ABR is measured at the irradiation frequency group consisting of 4 kHz, 8 kHz, 12 kHz, 16 kHz, and 32 kHz, in order to check whether the hearing threshold of the experimental object is changed or not. The measurements are sequentially performed from the low frequency level. Before noise exposure, ABR is measured as a control value, in order to check whether or not the experimental object may act in a normal range. Then, after the noise exposure, ABR is measured to record changes in the hearing threshold after the first, fifth, tenth, and twelfth laser irradiations.

SEM (Scanning Electron Microscope) Research

After the consecutive 12-day irradiation, the ABR result of the experimental object is recorded, and the experimental object is sacrificed in a state in which it is anesthetized. Then, a cochlea is secured from the experimental object. The secured cochlea is dipped in a 2% glutaraldehyde solution through the night, and rinsed by 0.1M PBS.

After dewatering is performed by graded ethanol, a critical point dryer is used to completely dewater the cochlea sample. The prepared cochlea sample is attached to an aluminum stub, and a target assembly is used to coat the cochlea sample with platinum-palladium. The surface of a basilar membrane having auditory hair cells is investigated by S-430 SEM.

In addition to the morphometric analysis, the cochlea may be analyzed in quality by recording the number of remaining auditory hair cells. The cochlea is divided into peak conversion, intermediate conversion, and base conversion at about 0° to 270°, 270° to 540°, and 540° to 810°, respectively. The number of auditory hair cells is measured at three different sites under a magnification of 600 times. The auditory hair cells are considered to be absent when a bundle of stereocilia is lost.

Statistical Analysis

All of the data is statistically analyzed by analysis of variance (ANOVA) based on statistical package for social science (SPSS). The data is expressed as ‘average±SD’, and differences are considered to be statistically important when p<0.05.

Hearing Threshold Change

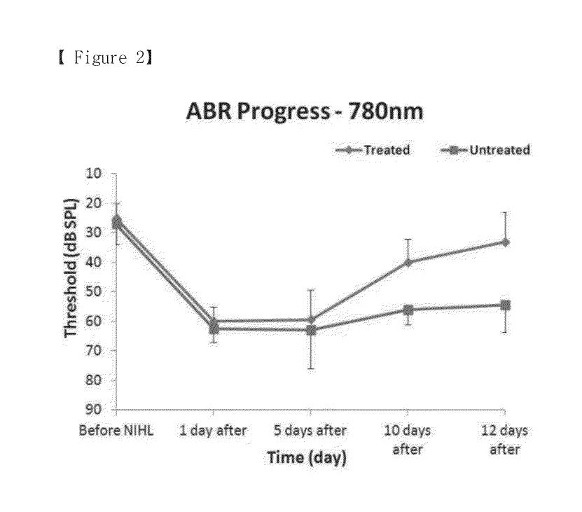

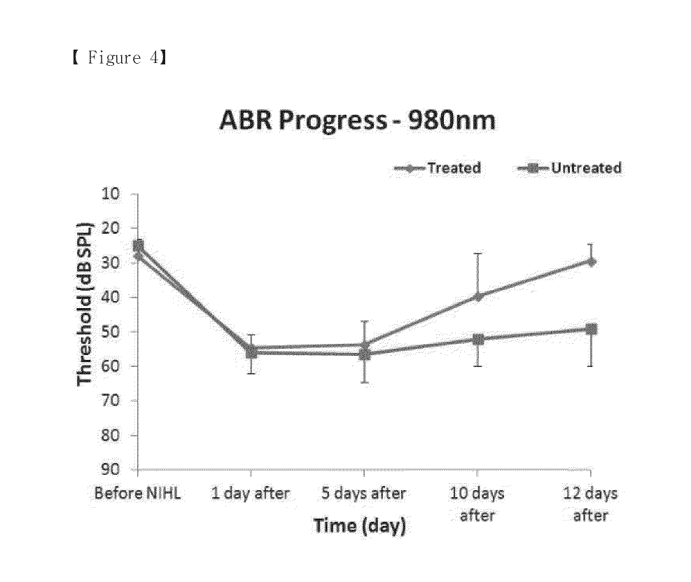

First, referring to FIGS. 2 to 4, changes of the hearing thresholds for the experimental objects onto which three representative lasers, that is, 780 nm, 830 nm, and 980 nm lasers are irradiated will be described.

Before noise-induced hearing loss (NIHL), the hearing thresholds of a treated group and an untreated group are maintained at 26 dB. After noise exposure, the hearing thresholds gradually decrease from about 60 dB till 5 days. As a whole, the treated group and the untreated, group have a small difference in hearing threshold therebetween.

Meanwhile, the hearing threshold of the treated group is significantly restored while laser irradiation is performed for 5 days to 12 days. On the other hand, the hearing threshold of the untreated group is not significantly changed during the period.

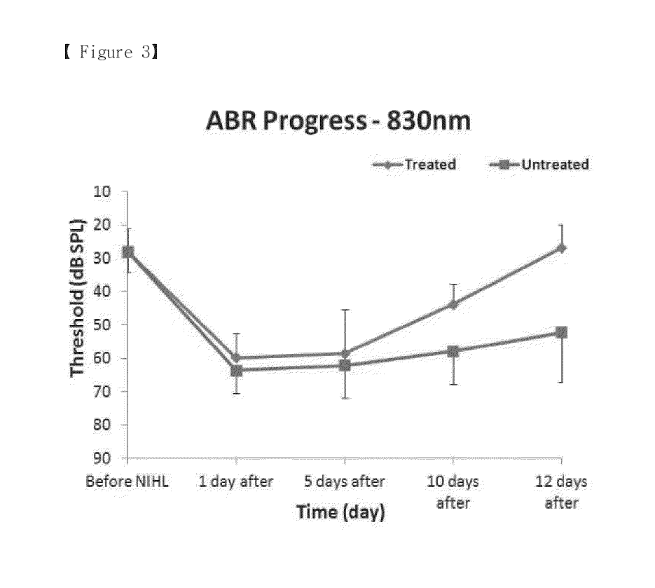

In particular, referring to FIG. 3, it can be seen that the hearing threshold of the treated group onto which the 830 nm laser is irradiated is restored, to the state before NIHL in 12 days after noise exposure.

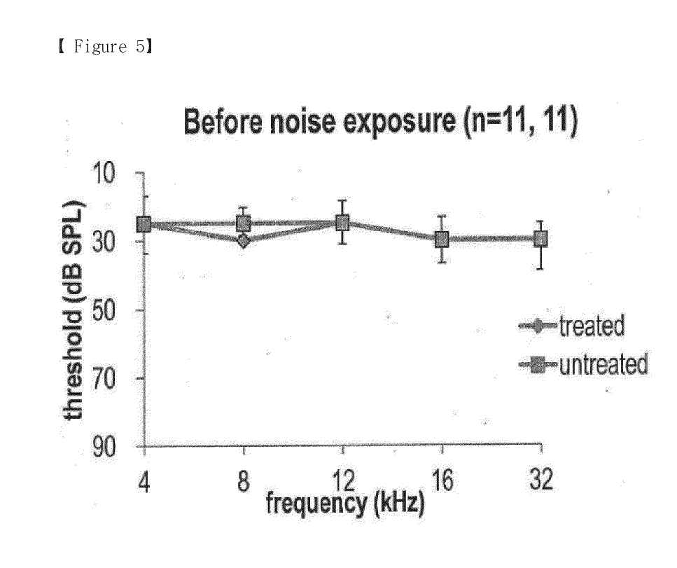

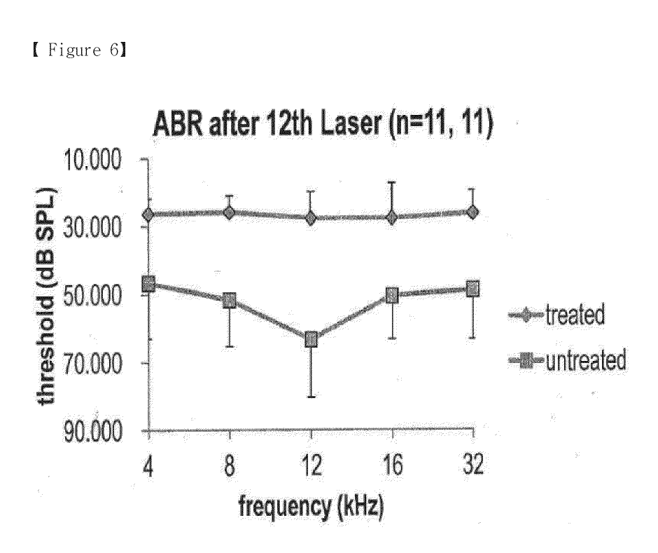

Meanwhile, referring to FIGS. 5 and 6, the hearing threshold, data before the experimental object is exposed to noise and the hearing threshold, data after the laser-irradiation is performed according to the embodiment of the present invention will be compared for each frequency band.

Before noise exposure as illustrated in FIG. 5, the hearing threshold of the experimental object is recorded as 26.5±4.7, 24.5±5.0, 24.0±5.2, 24.0±3.2, and 24.5±5.5 dB at frequencies of 4 kHz, 8 kHz, 12 kHz, 16 kHz, and 32 kHz, respectively. Immediately after noise exposure, ABR is recorded as 58.3±8.7, 62.8±3.6, 76.7±6.1, 60.6±6.8, and 60.0±9.0 dB SPL. That is, a significant increase occurs.

After 3 to 5-day long laser irradiation and 12th laser irradiation, it is recorded that the hearing threshold, of the treated group is significantly restored to 26.5±4.5, 25.9±4.9, 27.7±7.9, 27.7±10.3, and 26.4±6.7 dB SPL (p<0.05) as illustrated in FIG. 6. On the other hand, the hearing threshold of the untreated group is measured at 46.7±16.2, 51.7±13.7, 63.3±16.9, 50.6±12.6, and 48.9±14.3 dB SPL, after 12-day laser irradiation.

All of ABRs are arranged in order of 4 kHz, 8 kHz, 12 kHz, 16 kHz, and 32 kHz. Furthermore, for accurate measurement, the ABRs are averaged for the untreated group and the treated group in which 11 ears are laser-treated.

Morphological Variance and Auditory Hair Cell Survival

The cochlea is divided into peak conversion, intermediate conversion, and base conversion, and the numbers of auditory hair cells are accurately measured for each conversion at three different sites under a magnification of 600 times. The numbers of auditory hair cells across the basilar membrane of 200 μm are averaged for each group, in order to perform comparison. A control group composed of a total of 6 ears and a laser group composed, of a total of 16 ears are included in this experiment.

The numbers of auditory hair cells for peak conversion, intermediate conversion, and base conversion in an experimental object group which is not exposed to noise and laser-treated are measured at 176.5±16.3, 146±5.6, and 154.8±6.7, respectively, the numbers of auditory hair cells for peak conversion, intermediate conversion, and base conversion in an experimental object group which is only exposed to noise are measured at 122.5±2.1, 113.5±2.1, and 116.2±19.5, respectively, and the numbers of auditory hair cells for peak conversion, intermediate conversion, and base conversion in an experimental object group which is exposed to noise and laser-treated are measured at 167.4±27.5, 140.4±29.7, and 129.3±26.7, respectively. According to the result of the experiment, it can be seen that the experimental object group which is exposed to noise and laser-treated restores a large number of auditory hair cells in comparison to the experimental object group which is only exposed to noise.

Although a morphological difference in auditory hair cells is not as clear as ototoxicity induced by an ototoxicitic substance, it can be shown that a distortion and abnormality occur in stereocilia of the experimental object group which is only exposed to noise.

According to the method for low-level laser irradiation for hearing loss reversal, low-frequency laser light having a specific frequency is transmitted to the hearing-related organ positioned, in the inner ear through the external auditory meatus and the eardrum, and cures auditory hair cells, thereby lowering the hearing threshold.

Furthermore, when the method for low-level laser irradiation for hearing loss reversal is used, it is possible to expect a permanent treatment effect, compared to a conventional simple hearing aid.

While the present invention has been described with respect to the specific embodiments, it will be apparent to those skilled in the art that various changes and modifications may be made without departing from the spirit and scope of the invention as defined in the following claims.

What is claimed is:

1. A method for low-level laser irradiation for hearing loss reversal, comprising:
generating low-frequency laser light;
controlling an output intensity of the generated laser light;
determining whether or not the wavelength and output of the laser light coincides with a preset reference wavelength and reference output intensity, respectively; and
irradiating the laser light into an inner ear through an external auditory meatus,
wherein the reference wavelength is set in the range of 600 nm to 10,000 nm.

2. The method of claim 1, wherein the reference wavelength comprises any one selected from an infrared light wavelength group consisting of 780 nm, 830 nm, and 980 nm.

3. The method of claim 1, wherein the reference output intensity is set in the range of 50 mW to 300 mW.

4. The method of claim 3, wherein the reference output intensity is set to 165 mW.

5. The method of claim 1, wherein the irradiation of the laser light comprises periodically irradiating with the generated laser light for 10 to 60 minutes per day.

6. The method of claim 5, wherein irradiating with the laser light comprises irradiating with the generated laser light for 1 to 14 days.

7. The method of claim 1, further comprising checking a hearing threshold at a plurality of frequencies, after the laser light irradiation.

8. The method of claim 7, wherein the checking of the hearing threshold comprises checking a hearing threshold of a small animal at one or more irradiation frequencies selected from an irradiation frequency group consisting of 4 kHz, 8 kHz, 12 kHz, 16 kHz, and 32 kHz or checking a hearing threshold for a human body at one or more irradiation frequencies selected from an irradiation frequency group consisting of 0.25 kHz, 0.5 kHz, 1 kHz, 2 kHz, 4 kHz, and 8 kHz.
